(19) United States
(12) Patent Application Publication
(10) Pub. No.: US 2022/0233465 A1
(43) Pub. Date: Jul. 28, 2022

(54) CANNABINOID-COMPRISING COMPOSITIONS FOR MANAGEMENT OF PAIN

(71) Applicant: CANOPY GROWTH CORPORATION, Smiths Falls (CA)

(72) Inventor: Mark HETHERINGTON, Smiths Falls (CA)

(21) Appl. No.: 17/612,100

(22) PCT Filed: Apr. 29, 2020
(86) PCT No.: PCT/CA2020/050561

§ 371 Date: Nov. 17, 2021

Related U.S. Application Data
(60) Provisional application No. 62/851,421, filed on May 22, 2019.
(60) Provisional application No. 62/923,720, filed on Oct. 21, 2019.
(60) Provisional application No. 62/947,133, filed on Dec. 12, 2019.

(51) Int. Cl. A61K 31/05; A61K 36/28; A61K 36/324; A61K 31/045; A61K 31/165; A61K 31/015; A61K 31/125; A61K 9/00; A61P 29/00; A61K 9/06

(52) U.S. Cl. CPC A61K 31/05; A61K 36/28; A61K 36/324; A61K 31/045; A61K 9/06; A61K 31/015; A61K 31/125; A61K 9/0014; A61P 29/00; A61K 31/165

(57) ABSTRACT

There are provided compositions comprising a purified cannabidiol (CBD) or a derivative thereof and an anti-inflammatory botanical extract, and methods of use thereof for the management of pain in a subject.

### FIELD OF TECHNOLOGY

The present technology generally relates to compositions comprising cannabinoids and to methods of using same for the management of pain in a subject.

### BACKGROUND INFORMATION

Natural or synthetic cannabinoid receptor agonists have been shown to be of therapeutic value for a number of important medical conditions, including pain (e.g., against pain of neuropathic origin), anxiety, glaucoma, nausea, emesis, muscle spasms, and wasting diseases. Insofar as pain is concerned, it is known that cannabinoid receptor agonists have antinociceptive and anti-hyperalgesic effects at the peripheral and central (spinal and supraspinal) levels, as has been demonstrated in acute and chronic pain models.

Cannabinoid receptors and endocannabinoids are present in pain circuits from the peripheral sensory nerve endings up to the brain. Cannabinoid receptor agonists modulate nociceptive thresholds by regulating neuronal activity, but they also relieve pain by acting on non-nervous tissues. CB₁ receptor is involved in the attenuation of synaptic transmission, and a proportion of the peripheral analgesic effect of endocannabinoids can be attributed to a neuronal mechanism acting through CB₁ receptors expressed by primary afferent neurons. Although CB₂ receptors have been related traditionally to the peripheral effects of cannabinoids (mainly modulation of the immunologic responses), they also contribute to antinociception by inhibiting the release of proinflammatory factors by non-neuronal cells located near nociceptive neuron terminals. CB₂ receptors are expressed in several types of inflammatory cells and immunocompetent cells. For that reason, activation of peripheral CB₂ receptors generates an antinociceptive response in situations of inflammatory hyperalgesia and neuropathic pain.

The use of cannabinoids to alleviate pain has been proposed and tested. However, there remain many challenges associated with achieving therapeutic effectiveness of cannabinoid agonist-based medications in the management of various types of pain. There also remain many challenges with providing adequate and reproducible dosages of cannabinoids which are efficient in the management of pain.

### SUMMARY OF DISCLOSURE

According to various aspects, the present technology relates to a composition comprising a purified cannabidiol (CBD) or a derivative thereof and an anti-inflammatory botanical extract. In some instances, the composition is in a form suitable for topical administration to a subject.

According to various aspects, the present technology relates to a composition comprising a purified cannabidiol (CBD) or a derivative thereof; at least one purified terpene or a derivative thereof; and an anti-inflammatory botanical extract. In some instances, the composition is in a form suitable for topical administration to a subject. In some other instances, at least one purified terpene or the derivative thereof has anti-inflammatory properties, analgesic properties or both.

According to various aspects, the present technology relates to a method for management of pain in a subject. The method comprises administering to the subject, the composition as defined herein.

According to various aspects, the present technology relates to a method for alleviating pain in a subject. The method comprises administering to the subject, the composition as defined herein.

According to various aspects, the present technology relates to a method for treating pain in a subject. The method comprises administering to the subject, the composition as defined herein.

Other aspects and features of the present disclosure will become apparent to those ordinarily skilled in the art upon review of the following description of specific embodiments.

### DETAILED DISCLOSURE OF EMBODIMENTS

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which variations and additions do not depart from the present technology. Hence, the following description is intended to illustrate some particular embodiments of the technology, and not to exhaustively specify all permutations, combinations and variations thereof.

As used herein, the singular form “a,” “an” and “the” include plural referents unless the context clearly dictates otherwise.

The recitation herein of numerical ranges by endpoints is intended to include all numbers subsumed within that range (e.g., a recitation of 1 to 5 includes 1, 1.25, 1.5, 1.75, 2, 2.45, 2.75, 3, 3.80, 4, 4.32, and 5).

The term “about” is used herein, explicitly or not; every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. The term “or” as used herein should in general be construed non-exclusively. For example, an embodiment of “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

As used herein, the term “comprise” is used in its non-limiting sense to mean that items following the word are included, but items not specifically mentioned are not excluded.

As used herein, the term “wt %” means weight percent, which is also written herein as “w/w”. Weight percent is the weight of solute divided by the weight of solvent and multiplied by 100, to give the percent of solute in the solution. For example, 25 wt % of a compound in water means there is 25 grams of the compound for every 100 grams of water.

As used herein, the term “purified” means extracted, isolated, and/or separated from other compounds, formulations, compositions, matter, and/or mass resulting in a greater than 60% purity. In some embodiments a “purified” cannabinoid (or “purified” terpene) is greater than about 70% pure, greater than 75% pure, greater than about 80% pure, greater than 85% pure, greater than about 90% pure, greater than about 91% pure, greater than about 92% pure, greater than about 93% pure, greater than about 94% pure, greater than about 95% pure, greater than about 96% pure, greater than about 97% pure, greater than about 98% pure, or greater than about 99% pure. Within the context of the present disclosure, where a compound comprises stereogenic centers, the term “purified” includes enantiomerically pure compositions and also mixtures of enantiomers or isomers. Also within the context of the present disclosure, purified compounds may be purposely formulated with other compounds at various levels of purity. Provided that the ingredients used for purposeful formulation are purified prior to the said purposeful formulation, the act of subsequently formulating them does render them not “purified” within the context of an ingredient list. In an embodiment, the term “purified” may refer to a cannabinoid that is separated from plant matter from which it was derived. In an embodiment, the term “purified” may refer to a terpene that is separated from plant matter from which it was derived.

As used herein, the term “Cannabis” refers to the genus of flowering plants in the family Cannabaceae. Three species may be recognized as being part of the Cannabis genus, namely: Cannabis sativa, Cannabis indica, and Cannabis ruderalis. The expressions “Cannabis sativa” and “C. sativa” are used herein interchangeably. The term “strain” as used herein refers to different varieties of the plant genus Cannabis. For example, the term “strain” can refer to different pure or hybrid varieties of Cannabis plants. In some instances, the Cannabis strain of the present technology can by a hybrid of two strains, for example, a hybrid between C. sativa and C. indica. Different Cannabis strains often exhibit distinct chemical compositions with characteristic levels of cannabinoids and terpenes, as well as other components. Differing cannabinoid and terpene profiles associated with different Cannabis strains can be useful for the treatment of different diseases, or for treating different subjects with the same disease.

As used herein, the term “cannabinoid” refers to a chemical compound belonging to a class of secondary compounds commonly found in plants of genus Cannabis, but also encompasses synthetic and semi-synthetic cannabinoids.

As used herein, the expression “effective amount” or “therapeutically effective amount” refers to the amount of components of the compositions of the present disclosure which are effective for producing some desired therapeutic effect as defined herein at a reasonable benefit/risk ratio applicable to any treatment.

In one embodiment, the present technology relates to compositions comprising purified cannabidiol (CBD) or a derivative thereof and an anti-inflammatory botanical extract. In some implementations of this embodiment, the compositions of the present disclosure are useful in the management of pain in a subject (e.g., human and animal subjects).

i) Purified Cannabidiol (CBD)

CBD is one of the active cannabinoids identified in Cannabis. It is a major phytocannabinoid, by some accounts making up to 40% of the plant's extract. CBD does not appear to have any intoxicating effects such as those caused by THC in marijuana. CBD may be extracted from Cannabis plants or parts thereof using various solvents and technologies including, but not limited to, ethanol, butane, methane, heptane, carbon dioxide, ice, water, and steam. CBD may be extracted from Cannabis plants bred to express desired CBD profiles for purity. In some implementations, CBD may be extracted using supercritical fluid (SFC) extraction and similar technologies. In some instances, CBD is crystalized. The process of crystallization involves placing the compound of interest in a liquid and then cooling or adding participants to the solution which would lower the solubility of the compound of interest so that it forms crystals. In this example, crystals are then separated from the liquid through filtration or centrifugation.

In some embodiments, the CBD useful in the compositions of the present technology is synthetic or biosynthetic CBD.

Derivatives of cannabidiol that may be used in the compositions of the present technology include compounds that can be imagined to arise or actually be synthesized from a cannabidiol by replacement of one atom of its structure with another atom or with a group of atoms. An example of a derivative of CBD is cannabidiol dimethyl heptyl (CBD-DMH), including all isomeric forms thereof.

In some embodiments, CBD or a derivative thereof is present in the compositions of the present technology in an amount ranging from between about 1 wt % and about 10 wt % of the total weight of the composition. In some instances, CBD or a derivative thereof is present in the compositions of the present technology in an amount ranging from between about 2 wt % and about 8 wt %, or between about 2 wt % and about 6 wt %, or between about 2.5 wt % and about 5 wt %, or is present in an amount of about 2.5 wt %, or is present in an amount of about 5 wt %, or is present in an amount of about 6 wt % of the total weight of the composition.

In some embodiments, the compositions of the present technology comprise an amount of one or more cannabinoids other than CBD. In some instances, the minimal amounts of other cannabinoids range between about 0.01 wt % and about 5.0 wt % of the total weight of the composition. The cannabinoids other than CBD may include one or more of: cannabigerol ((E)-CBG C-5), cannabigerol monomethyl ether ((E)-CBGM C-5A), Cannabinerolsäure A ((Z)-CBGA C-5A), Cannabigerovarin (((e)-CBGV C-3), Cannabigerolsäure A (e)-CBGA C-5A), A Cannabigerolsäure monomethyl ether ((e)-CBGAM C-5A), Cannabigerovarinsäure A ((e)-CBGVA-C3A); cannabichromene (CBC-C5), Cannabichromensäure A (CBCA C-5A), Cannabichromevarin (CBCVC-3), Cannabichromevarinsäure A (CBCVA-C3A); cannabidiol (CBD-C5), cannabidiol monomethyl (CBDM-C5), cannabidiol-C4 (CBD-C4), Cannabidivarin (CBDV-C3), Cannabidiorcol (CBD-C1), cannabidiolic (CBDA C-5), Cannabidivarinsäure (CBDVA C-3); Cannabinodiol-like (CBND): Cannabinodiol (CBND C-5), Cannabinodivarin (CBND C-3); Tetrahydrocannabinol-like (THC): Δ⁹-tetrahydrocannabinol (Δ⁹-THC-C5), Δ⁹-tetrahydrocannabinol-C4 (Δ⁹-THC-C4), Δ⁹-tetrahydrocannabivarin (Δ⁹-THCV-C3), Δ⁹-Tetrahydrocannabiorcol (Δ⁹-THCO C-1), Δ⁹-Tetrahydrocannabinolsäure (Δ⁹THCA-C-5A), Δ⁹-Tetrahydrocannabinolsäure B (Δ⁹THCA-C-5B), Δ⁹-Tetrahydrocannabinolsäure-C4 (Δ⁹THCA-C-4A and/or B), Δ⁹-Tetrahydrocannabivarinsäure A (Δ⁹-THCVA-C3A), Δ⁹-Tetrahydrocannabiorcolsäure (Δ⁹-THCOA-C1A and/or B), (−)-Δ⁸-trans-(6aR,10aR)-Δ⁸-tetrahydrocannabinol (Δ⁸-THC-C5), (−)-Δ⁸-trans-(6aR,10aR)-Tetrahydrocannabinolsäure A (Δ⁸-THCA-C5A); (−)-(6aS,10aR)-Δ⁹-tetrahydrocannabinol ((−)-cis-Δ⁹-THC-C5); Cannabinol CBN-C5, cannabinol C4 (CBN-C4), Cannabivarin (CBN-C3), cannabinol C2 (CBN-C2), Cannabiorcol (CBN-C1), Cannabinolsäure A (C5 CBNA-A), Cannabinolmethylether (CBNM C-5) Cannabitriol-type (CBT): (−)-(9R,10R)-trans-Cannabitriol ((−)-trans-CBT-C5), (+)-(9S,10S)-Cannabitriol ((+)-trans-CBT C-5), (i)-(9R,10S/9S,10R)-Cannabitriol ((i)-cis-CBT-C5), (−)-(9R,10R)-trans [10-O-ethyl-cannabitriol] ((−)-trans-CBT-OEt-C5), (i)-(9R,10R/9S, 10S)-Cannabitriol-C3 ((i)-trans-CBT-C3), 8,9-dihydroxy-Δ6a (10a) tetrahydrocannabinol (8,9-di-OH-CBT-C5), cannabidiolic A (CBDA C-59-OH-CBT-C5 ester), (−)-(6aR,9S,10S,10aR)-9,10-dihydroxy-hexahydrocannabinol, Cannabiripsol Cannabiripsol-C5, (−)-6a,7,10α-trihydroxy-Δ⁹-tetrahydrocannabinol ((−)-Cannabitetrol), 10-oxo-Δ6a (10a) tetrahydrocannabinol (OTHC); Cannabielsoin-like (CBE): (5aS,6S,9R,9aR)-C5-Cannabielsoin (CBEC-5), (5aS,6S,9R,9aR)-C3-Cannabielsoin (CBE C-3), (5aS,6S,9R,9aR)-Cannabielsoinsäure A (CBEA-C5 A), (5aS,6S,9R,9aR)-Cannabielsoinsäure B (CBEA-C5 B), (5aS,6S, 9R,9aR)-C3-Cannabielsoinsäure B (CBEA-C3B), Cannabiglendol-C3 (OH-iso-HHCV C-3), Dehydrocannabifuran (DCBF C-5), Cannabifuran (CBF-C5); Isocannabinoide: (−)-Δ7-trans-(1R, 3R, 6R)-Isotetrahydrocannabinol, (−)-Δ7-1,2-cis-(1R, 3R, 6S/1S, 3S, 6R)-Isotetrahydrocannabivarin, (−)-Δ7-trans-(1R,3R,6R)-Isotetrahydrocannabivarin; (−)-(1aS,3aR,8bR,8Cr-cannabicyclol (CBL-C5), (−)-(1aS, 3aR, 8bR,8Cr-Cannabicyclolsäure A (CBLA-C5A) (−)-(1aS,3aR,8bR,8Cr-Cannabicyclovarin (CBLV C-3); Cannabicitran-type (CBT): Cannabicitran (CBT-C5); Cannabichromanon (CBCN C-5), Cannabichromanon-C3 (CBCN C-3), Cannabicoumaronon (CBCON C-5).

In some implementations, the compositions of the present technology may comprise THC, CBD, CBG, CBN, CBC, THCV, CBGA, CGCA, CBCA, THCA and CBDA. In some implementations, the compositions of the present technology comprise THC. In an embodiment, the compositions of the present technology comprise from about 0.1 wt % to about 2 wt % THC, from about 0.25 wt % to about 2 wt % THC, from about 0.5 wt % to about 5 wt % THC, or comprises about 0.125 wt %, or about 0.5 wt %, or about 1.0 wt % or about 1.2 wt %, or about 1.5 wt %, or about 2.0 wt % of a THC distillate (85%).

In some embodiments, the compositions of the present technology may comprise CBDA or a derivative thereof. In some implementations of these embodiments, CBDA or a derivative thereof is present in the compositions of the present technology in an amount ranging from between about 0.01 wt % and about 10 wt % of the total weight of the composition. In some instances, CBDA or a derivative thereof is present in the compositions of the present technology in an amount ranging from between about 1 wt % and about 10 wt %, or between about 2 wt % and about 8 wt %, or between about 3 wt % and about 6 wt %, or between about 4 wt % and about 6 wt %, or is present in an amount of about 5 wt % or is present in an amount of about 6 wt % of the total weight of the composition.

ii) Purified Terpenes

As used herein, the term “terpene” refers to an organic compound built on an isoprenoid structural scaffold or produced by combining isoprene units. Often, terpene molecules found in plants may produce smell. The structures of terpenes are built with isoprenes, which are 5 carbon structures. Flavonoids are generally considered to be 15 carbon structures with two phenyl rings and a heterocyclic ring. So, there could be an overlap in which a flavonoid could be considered a terpene. However, not all terpenes could be considered flavonoids.

Within the context of the present technology, terpenes and derivatives thereof include, but are not limited to: hemiterpenes, monoterpenols, terpene esters, diterpenes, monoterpenes, polyterpenes, tetraterpenes, terpenoid oxides, sesterterpenes, sesquiterpenes, norisoprenoids, and their derivatives. Derivatives of terpenes that may be used in the compositions of the present technology include compounds that can be imagined to arise or actually be synthesized from a terpene by replacement of one atom of its structure with another atom or with a group of atoms. Derivatives of terpenes include terpenoids in the form of hemiterpenoids, monoterpenoids, sesquiterpenoids, sesterterpenoids, sesquarterpenoids, tetraterpenoids, triterpenoids, tetraterpenoids, polyterpenoids, isoprenoids, and steroids. They may be in the form of α-, β-, γ-, and oxo-isomers, or combinations thereof.

Further examples of terpenes or derivatives thereof include, but are not limited to: 7,8-dihydroionone, Acetanisole, Acetic Acid, Acetyl Cedrene, Anethole, Anisole, Benzaldehyde, Bergamotene (α-cis-Bergamotene) (α-trans-Bergamotene), Bisabolol (α-Bisabolol), Borneol, Bornyl Acetate, Butanoic/Butyric Acid, Cadinene (α-Cadinene) (γ-Cadinene), Cafestol, Caffeic acid, Camphene, Camphor, Capsaicin, Carene (Δ-3-Carene), Carotene, Carvacrol, Carvone, Dextro-Carvone, Laevo-Carvone, Caryophyllene (β-Caryophyllene), Caryophyllene oxide, Castoreum Absolute, Cedrene (α-Cedrene) (β-Cedrene), Cedrene Epoxide (α-Cedrene Epoxide), Cedrol, Cembrene, Chlorogenic Acid, Cinnamaldehyde (α-amyl-Cinnamaldehyde) (α-hexyl-Cinnamaldehyde), Cinnamic Acid, Cinnamyl Alcohol, Citronellal, Citronellol, α-Copaene, Cryptone, Curcumene (α-Curcumene) (γ-Curcumene), Decanal, Dehydrovomifoliol, Diallyl Disulfide, Dihydroactinidiolide, Dimethyl Disulfide, Eicosane/lcosane, Elemene (β-Elemene), Estragole, Ethyl acetate, Ethyl Cinnamate, Ethyl maltol, Eucalyptol/1,8-Cineole, Eudesmol (α-Eudesmol) (β-Eudesmol) (γ-Eudesmol), Eugenol, Euphol, Farnesene, Farnesol, Fenchol (β-Fenchol), Fenchone, Geraniol, Geranyl acetate, Germacrenes, Germacrene B, Guaia-1 (10),11-diene, Guaiacol, Guaiene (α-Guaiene), Gurjunene (α-Gurjunene), Herniarin, Hexanaldehyde, Hexanoic Acid, Humulene (α-Humulene) (β-Humulene), lonol (3-oxo-α-ionol) (β-IoηoI), lonone (α-lonone) (-lonone), Ipsdienol, Isoamyl acetate, Isoamyl Alcohol, Isoamyl Formate, Isoborneol, Isomyrcenol, Isopulegol, Isovaleric Acid, Isoprene, Kahweol, Lavandulol, Limonene, γ-Linolenic Acid, Linalool, Longifolene, α-Longipinene, Lycopene, Menthol, Methyl butyrate, 3-Mercapto-2-Methylpentanal, Mercaptan/Thiols, β-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (β-Myrcene), γ-Muurolene, Nepetalactone, Nerol, Nerolidol, Neryl acetate, Nonanaldehyde, Nonanoic Acid, Ocimene, Octanal, Octanoic Acid, P-cymene, Pentyl butyrate, Phellandrene, Phenylacetaldehyde, Phenylethanethiol, Phenylacetic Acid, Phytol, Pinene, β-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, α-Selinene, α-Sinensal, β-Sinensal, β-Sitosterol, Squalene, Taxadiene, Terpin hydrate, Terpineol, Terpine-4-ol, α-Terpinene, γ-Terpinene, Terpinolene, Thiophenol, Thujone, Thymol, α-Tocopherol, Tonka Undecanone, Undecanal, Valeraldehyde/Pentanal, Verdoxan, α-Ylangene, Umbelliferone, and Vanillin.

In some embodiments, the terpene or derivative thereof is bisabolol which includes either or both of the β and/or α forms of a monocyclic sesquiterpene alcohol in a pure form and/or a mixture of any ratio. Bisabolol is often considered as having a medium strength, citrus, floral, tangy, lemon, fresh, sweet, or herbaceous aroma. It has shown anti-inflammatory, antimicrobial, antifungal and/or antimutagenic activities. In such embodiments, the composition of the present technology thus comprises CBD and bisabolol.

In some other embodiments, the terpene or derivative thereof is cadinene which includes either or both of α-cadinene and/or γ-cadinene as pure forms or mixtures in any ratio. α-Cadinene refers to an isomeric hydrocarbon sesquiterpene. α-Cadinene is often considered as having a pungent, smoky, woody, guaiac or wood-like odor. It has shown some antimicrobial, anticancer, anti-inflammatory, antioxidant and/or antimalarial activities. In such embodiments, the composition of the present technology thus comprises CBD and cadinene.

In some other embodiments, the terpene or derivative thereof is camphor. Camphor has a characteristic odor for which the tree is named. The most recognizable Camphor product has a strong, penetrating, and often persistent odor. The therapeutic properties of camphor oil are analgesic, antidepressant, anti-inflammatory, antiseptic, cardiac, carminative, diuretic, febrifugal, hypertensive, insecticidal, laxative, rubefacient, stimulatory, sudorific, vermifugal and/or vulnerary. In such embodiments, the composition of the present technology thus comprises CBD and camphor.

In some other embodiments, the terpene or derivative thereof is a capsaicinoid, such as capsaicin. Capsaicin is often used as an analgesic in topical ointments and dermal patches to relieve pain via several pathways including substance P modulation and/or as an anti-inflammatory. In such embodiments, the composition of the present technology thus comprises CBD and capsaicin. In some embodiments, the composition of the present technology comprises CBD and a capsaicinoid.

In some other embodiments, the terpene or derivative thereof is carvacrol. Carvacrol is often considered as having a pungent, warm odor of oregano. It is used as a flavor and fragrance agent and its flavor has been described as spicy, herbal, phenolic, medicinal and woody. Carvacrol exhibits many diverse activities such as: antimicrobial, antitumor, antimutagenic, antigenotoxic, analgesic, antispasmodic, anti-inflammatory, angiogenic, antiparasitic, antiplatelet, AChe inhibitory, antielastase, insecticidal, antihepatotoxic and/or hepatoprotective activities. In such embodiments, the composition of the present technology thus comprises CBD and carvacrol.

In some other embodiments, the terpene or derivative thereof is copaene, in particular, α-copaene. Copaene is an oily liquid hydrocarbon (copaiba balsam/copaifera) that is found in a number of essential oil-producing plants. The name is derived from that of the resin-producing tropical copaiba tree. In such embodiments, the composition of the present technology thus comprises CBD and copaene (e.g., copaiba balsam). In such embodiments, the composition of the present technology thus comprises CBD, THC and copaene (e.g., copaiba balsam).

In some other embodiments, the terpene or derivative thereof is caryophyllene or beta-caryophyllene. Caryophyllene, or -β-caryophyllene, is a natural bicyclic sesquiterpene that is a constituent of many essential oils, especially clove the oil from the stems and flowers of Syzygium aromaticum, the essential oil of Cannabis sativa, rosemary, hops, and copaifera oil. In such embodiments, the composition of the present technology thus comprises CBD and caryophyllene or beta-caryophyllene.

In some other embodiments, the terpene or derivative thereof is farnesol. Farnesol is often considered as having a weak, mild, fresh, sweet, floral, and/or linden tree odor. It is used as an agent in cosmetics, flavors and fragrances. It has anti-inflammatory, antioxidant and/or antiproliferative effects and it has been suggested to function as a chemopreventive and antitumor agent with some analgesic potential. Farnesol is present in many essential oils such as citronella, neroli, cyclamen, lemon grass, tuberose, rose, musk, balsam and tolu. In such embodiments, the composition of the present technology thus comprises CBD and farnesol.

In some other embodiments, the terpene or derivative thereof is guaia-1 (10),11-diene. Guaia-1 (10),11-diene is often considered as having an elegant and sweet woody aroma. As used herein, the term guaiacol refers to an organic compound with a phenol group with a methoxy group in the ortho-position. Guaiacol is often characterized as having a powerful, smoke-like, phenolic, spicy, woody and/or somewhat medicinal odor. It has a sweet, powdery, musty, vanilla, floral, and/or almond flavor. It is used chiefly as an expectorant, but it is also used as a local anesthetic, an antiseptic and an intestinal disinfectant. Guaiacol is a precursor to various flavorants, such as eugenol and vanillin. α-Guaiene is often considered as having a medium strength, sweet, earthy, woody, balsamic, or peppery aroma. In such embodiments, the composition of the present technology thus comprises CBD and guaia-1 (10), 11-diene.

In some other embodiments, the terpene or derivative thereof is humulene which includes either or both the α-Humulene and/or the β-Humulene isomers as pure forms or mixtures in any ratio. They are monocyclic sesquiterpenes with an 11-membered ring. α-Humulene (obsolete name: α-Caryophyllene) is often considered as having an aroma that has been described as bitter, medium woody, and/or hoppy. α-Humulene has shown anti-inflammatory properties. Humulene is one of the essential oils made in the flowering cone of the hops plant Humulus lupulus. In such embodiments, the composition of the present technology thus comprises CBD and humulene.

In some other embodiments, the terpene or derivative thereof is isoborneol which is an isomer of borneol with the alcohol group in a different position. Isoborneol is often considered as having a camphoraceous, sweet and/or musty, India ink-like aroma. It has shown antioxidant, anti-inflammatory and/or some limited antimicrobial properties. It is often considered as having a fresh, floral, or lime-like odor. In such embodiments, the composition of the present technology thus comprises CBD and isoborneol.

In some other embodiments, the terpene or derivative thereof is γ-linolenic acid. It is often considered as having the slight, light aroma of vegetable oil. This is because it is an unsaturated fatty acid found primarily in vegetable oils. It is used as a slight anti-inflammatory and/or blood-thinning agent. In such embodiments, the composition of the present technology thus comprises CBD and γ-linolenic acid.

In some other embodiments, the terpene or derivative thereof is methyl salicylate. It is often considered as having a strong, distinct wintergreen aroma. It is used as a fragrance, in foods and beverages, and in liniments where it is commonly used as a topical medicine for muscle, joint or soft tissue pain and/or inflammation. In such embodiments, the composition of the present technology thus comprises CBD and methyl salicylate.

In some other embodiments, the terpene or derivative thereof is β-sitosterol which refers to plant sterols with a similar structure to cholesterols. It is often considered as having a characteristic waxy aroma. It is found in pecans, avocados, pumpkin seeds, cashews, corn oils, and soybeans. It has shown anti-inflammatory properties. In such embodiments, the composition of the present technology thus comprises CBD and β-sitosterol.

In some other embodiments, the terpene or derivative thereof is stigmasterol. Stigmasterol is a plant sterol (phytosterol) and is among the most abundant of plant sterols, having a major function to maintain the structure and physiology of cell membranes. It may be used in food manufacturing to increase the phytosterol content, potentially lowering the levels of LDL cholesterol. In such embodiments, the composition of the present technology thus comprises CBD and stigmasterol.

In some other embodiments, the terpene or derivative thereof is campesterol. Campesterol is a phytosterol with a chemical structure similar to the one of cholesterol. In such embodiments, the composition of the present technology thus comprises CBD and campesterol.

In some other embodiments, the terpene or derivative thereof is brassicasterol. Brassicasterol (24-methyl cholest-5,22-dien-30-ol) is a 28-carbon sterol synthesised by several unicellular algae (phytoplankton) and some terrestrial plants, like rape. In such embodiments, the composition of the present technology thus comprises CBD and brassicasterol.

In some other embodiments, the terpene or derivative thereof is γ-terpinene. It is often considered as having a herbaceous, citrusy sweet aroma. It is commercially extracted from tea tree oil. γ-terpinene shows antimicrobial properties against various human pathogens. It has also been studied for its antioxidant, anti-inflammatory and/or antiproliferative activities. In such embodiments, the composition of the present technology thus comprises CBD and γ-terpinene.

In some other embodiments, the terpenes useful in the compositions of the present technology are oleoresins. As used herein, the term “oleroresin” refers to semi-solid extracts composed of a resin in solution in essential and/or fatty oil, obtained by evaporation of the solvent(s) used for their production. Naturally occurring oleoresins are also known as balsams. Oleoresins abound in heavier, less volatile and/or lipophilic compounds, such as resins, waxes, fats and fatty oils. Oleoresins are prepared from spices, such as basil, Capsicum (paprika), cardamom, celery seed, cinnamon bark, copaifera, clove bud, fenugreek, fir balsam, ginger, jambu, labdanum, mace, marjoram, nutmeg, parsley, pepper (black/white), pimenta (allspice), rosemary, sage, savory (summer/winter), thyme, turmeric, vanilla, and West Indian bay leaves. The solvents used are nonaqueous and may be polar (alcohols) or nonpolar (hydrocarbons, carbon dioxide). In such embodiments, the composition of the present technology thus comprises CBD and an oleoresin. In the instances where Capsicum is used in the compositions as defined herein, an additional agent may be incorporated into the composition to alleviate the heat/burn feeling that may be created by the Capsicum.

In some embodiments, the terpenes or derivatives thereof useful in the compositions of the present technology are extracted from Cannabis plants or parts thereof using various solvents and technologies including, but not limited to ethanol, butane, methane, carbon dioxide, ice, water, and steam. The terpenes may be extracted and/or purified from plants bred to express desired terpene profiles.

In some embodiments, the terpenes useful in the compositions of the present technology are synthetic terpenes.

In some embodiments, terpene or the derivative thereof is present in the compositions of the present technology in an amount of between about 0.25 wt % and about 20 wt %, or between about 0.25 wt % and about 10 wt %, or between about 0.5 wt % and about 20 wt %, or between about 0.5 wt % and about 10 wt %, or between about 1 wt % and about 20 wt %, or between about 1 wt % and about 10 wt % of the total weight of the composition. In some instances, terpene or the derivative thereof is present in the compositions of the present disclosure in an amount ranging from between about 0.25 wt % and about 8 wt %, or between about 1% and about 8%, or between about 2 wt % and about 8 wt %, or between about 3 wt % and about 6 wt %, or between about 4 wt % and about 6 wt %, or is present in an amount of about 2 wt %, or is present in an amount of about 5 wt % or is present in an amount of about 6 wt % of the total weight of the composition.

In some embodiments, the ratio of CBD or the derivative thereof and terpene or the derivative thereof (CBD:terpene) in the compositions of the present technology is about 1:0.25, or about 1:50, or about 1:0.75, or about 1:1, or about 1:1.25, or about 1:1.5, or about 1:1.75, or about 1:2, or about 1.25:1, or about 1.5:1, or about 1.75:1, or about 2:1, or about 3:1, or about 4:1, or about 5:1, or about 1:3, or about 1:4, or about 1:5.

iii) Anti-Inflammatory Botanical Extracts

In some embodiments, the anti-inflammatory botanical extracts useful in the compositions of the present technology include substances which are capable of imparting some anti-inflammatory effects when such substances are used in the compositions as defined herein and are administered to a subject. In some implementations of these embodiments, the anti-inflammatory botanical extracts of the present technology are also known for imparting analgesic effects when administered to a subject. As such, in those implementations, the anti-inflammatory botanical extracts of the present technology are also analgesic botanical extracts.

In the context of the present technology, the botanical extracts refer to a collection of crude mixtures extracted from plants or from parts of plants. In some implementations, the anti-inflammatory botanical extracts of the present technology are obtained through plant extraction processes which typically involve a solid/liquid separation operation wherein the plant or part thereof is placed in contact with a fluid (a solvent) and the plant components of interest are then solubilized and contained in the solvent. Methods for obtaining plant extracts are known to those of skill in the art.

Examples of anti-inflammatory plant extracts that be used in the composition as defined herein include, but are not limited to: Angelica extract, avocado extract, Tasmannia lanceolata extract, wild yam extract, Boswellia spp. extract, fenugreek extract, Harpagophytum spp. extract, hydrangea extract, althea extract, Arnica spp. extract, aloe extract (also referred to herein as aloe vera extract and aloe vera leaf extract), apricot extract, apricot core extract, ginkgo extract, fennel extract, turmeric extract, oolong tea extract, rose fruit extract, Echinacea leaf extract, Scutellaria root extract, Phellodendron bark extract, goldthread extract, barley extract, Hypericum extract, white nettle extract, watercress extract, orange extract, sea salt, seaweed extract, hydrolyzed elastin, hydrolyzed wheat powder, hydrolyzed silk, chamomile extract, carrot extract, Artemisia capillaris extract, Glycyrrhiza extract, Sabdariffa extract, Pyracantha fortuneana fruit extract, Cinchona extract, cucumber extract, guanosine, Gardenia extract, Sasa albo-marginata extract, Sophora root extract, walnut extract, grapefruit extract, Clematis extract, Chlorella extract, mulberry bark extract, gentian extract, black tea extract, yeast extract, burdock extract, fermented rice bran extract, rice germ oil, comfrey extract, collagen, cowberry extract, Asiasarum root extract, Bupleurum falcatum root extract, umbilical cord extract, salvia extract, saponaria extract, bamboo grass extract, Crataegus extract, Zanthoxylum fruit extract, shiitake mushroom extract, Rehmannia root extract, Lithospermum root extract, Perilla extract, linden extract, Filipendula extract, peony root extract, Calamus rhizome extract, birch extract, horsetail extract, ivy extract, hawthorn extract, Sambucus nigra extract, yarrow extract, peppermint extract, sage extract, mallow extract, Cnidium rhizome extract, Swertia herb extract, soy extract, jujube extract, wild thyme extract, green tea extract, clove extract, cogon extract, citrus unshiu peel extract, Angelica root extract, calendula extract, peach seed extract, bitter orange extract, Houttuynia extract, tomato extract, natto extract, Ginseng extract, garlic extract, wild rose extract, Hibiscus sabdariffa flower extract, Ophiopogon tuber extract, parsley extract, honey, witch hazel extract, pellitory extract, isodonis extract, Matricaria extract, loquat extract, coltsfoot extract, butterbur scape extract, Poria cocos extract, butcher bloom extract, grape extract, propolis, luffa extract, safflower extract, peppermint extract, linden extract, peony extract, hop extract, pine extract, horse chestnut extract, skunk cabbage extract, sapindaceae extract, balm mint extract, peach extract, cornflower extract, eucalyptus extract, saxifrage extract, Coix seed extract, mugwort extract, lavender extract, apple extract, lettuce extract, lemon extract, Chinese milk vetch extract, rose extract, rosemary extract, Roman and/or German chamomile extract and royal jelly extract.

In some embodiments, the anti-inflammatory botanical extract is present in the compositions of the present technology in an amount of between about 0.5 wt % to about 90 wt %, or between about 1 wt % and about 90 wt % of the total weight of the composition, or is present in an amount of about 5 wt % of the total weight of the composition.

In some instances, the anti-inflammatory botanical extract is present in an amount of between about 10 wt % and about 95 wt %, or between about 5 wt % and about 95 wt %, or between about 10 wt % and about 90 wt %, or between about 5 wt % and about 10 wt %, or between about 20 wt % and about 90 wt %, or between about 30 wt % and about 90 wt %, or between about 40 wt % and about 90 wt %, or between about 50 wt % and about 90 wt %, or between about 60 wt % and about 90 wt %, or between about 70 wt % and about 90 wt %, or between about 80 wt % and about 90 wt %, or is present in an amount of about 85 wt %.

In some embodiments, the compositions of the present technology comprise Angelica root extract. In some other embodiments, the compositions of the present technology comprise CBD and Angelica root extract. In some further embodiments, the compositions of the present technology comprise CBD and Angelica root extract, wild yam extract, and/or aloe extract or juice (e.g., aloe vera juice, aloe vera leaf juice, etc.).

In some embodiments, the compositions of the present technology comprise Tazman Pepper™, which is an anti-inflammatory agent extracted from the Tasmanian pepper fruit. In some other embodiments, the compositions of the present technology comprise CBD and Tazman Pepper™. In some further embodiments, the compositions of the present technology comprise CBD, Tazman Pepper™, copaiba balsam, Angelica root extract, vitamins (e.g., vitamin E), wild yam extract, capsaicinoids and/or aloe extract or juice (e.g., aloe vera juice, aloe vera leaf juice, etc.).

In some embodiments, the compositions of the present technology comprise Arnica oil, which has anti-inflammatory properties and can be used to treat bruises, muscle aches, muscle soreness, and muscle spasms. In some other embodiments, the compositions of the present technology comprise CBD and Arnica oil. In some further embodiments, the compositions of the present technology comprise CBD, Arnica oil, copaiba balsam, Boswellia extract, alpha-bisabolol, vitamins (e.g., vitamin E) and/or aloe extract or juice (e.g., aloe vera juice, aloe vera leaf juice, etc.).

In some embodiments, the compositions of the present technology comprise Boswellia extract, which is used as an anti-inflammatory agent and to alleviate pain. In some other embodiments, the compositions of the present technology comprise CBD and Boswellia extract. In some further embodiments, the compositions of the present technology comprise CBD, Boswellia extract, Arnica oil, copaiba balsam, alpha-bisabolol, vitamins (e.g., vitamin E) and/or aloe extract or juice (e.g., aloe vera juice, aloe vera leaf juice, etc.).

In some embodiments, the compositions of the present technology comprise CBD and copaiba balsam, THC, vitamins (vitamin E) and/or aloe extract or juice (e.g., aloe vera juice, aloe vera leaf juice, etc.).

In some embodiments, the compositions of the present technology comprise CBD and copaiba balsam, vitamins (e.g., vitamin E) and/or aloe extract or juice (e.g., aloe vera juice, aloe vera leaf juice, etc.).

iv) Non-Medicinal Ingredients

In some embodiments, the compositions of the present technology further comprise non-medicinal ingredients. The non-medical ingredients should be acceptable/suitable in the sense of being compatible with any other components in the composition and should not be deleterious to the subject. In some instances, the non-medical ingredients may be incorporated into the compositions defined herein with the purpose of, for example, improving administration to the subject, improving bioavailability of the components of the composition, adjusting pH of the composition, modifying the texture of the composition, improving the stability of the composition, and the like. Examples of non-medicinal ingredients useful in the compositions of the present technology include, but are not limited to: absorbents, abrasives, anticaking agents, antifoaming agents, antimicrobial agents, binders, biological additives, buffering agents, bulking agents, chemical additives, cosmetic biocides, denaturants, cosmetic astringents, drug astringents, external analgesics, film formers, humectants, opacifying agents, fragrances, perfumes, pigments, colorings, essential oils, skin sensates, emollients, skin soothing agents, skin healing agents, pH adjusters, plasticizers, preservatives, preservative enhancers, propellants, reducing agents, skin-conditioning agents, skin penetration enhancing agents, skin protectants, solvents, suspending agents, emulsifiers, thickening agents, solubilizing agents, stabilization agents, waxes, sunscreens, sunblocks, ultraviolet light absorbers or scattering agents, sunless tanning agents, antioxidants and/or radical scavengers, chelating agents, and sequestrants. Such other materials are known in the art.

In some such embodiments, the compositions of the present technology comprise Span™ 60, which is primarily used as an emulsifier and solubilizing agent. In some such embodiments, the compositions of the present technology comprise one or more polysorbate, which are also used as emulsifiers and solubilizing agents. For example, the compositions of the present technology may comprise Polysorbate 60 and/or Polysorbate 80. In some embodiments, the compositions of the present technology comprise Span™ 60, Polysorbate 60 and/or Polysorbate 80.

In some such embodiments, the compositions of the present technology comprise Polawax™ NF, which is a nonionic emulsifying wax made of a combination of vegetable-derived emulsifiers and stabilizers. In some embodiments, the compositions of the present technology therefore comprise CBD and Polawax™ NF.

In some such embodiments, the compositions of the present technology comprise EDTA Disodium Dihydrate, which is a chelating agent primarily used for its preservative, antimicrobial, and antioxidant properties. In some embodiments, the compositions of the present technology comprise CBD and EDTA Disodium Dihydrate.

In some such embodiments, the compositions of the present technology comprise phenoxyethanol (PE), which is primarily used as a preservative. In some embodiments, the compositions of the present technology therefore comprise CBD and phenoxyethanol (PE).

In some such embodiments, the compositions of the present technology comprise Ethylhexyl Glycerin (EHG), which is derived from glycerin and commonly found in animal fat and vegetable oil EHG is used primarily for its antimicrobial preservative properties and also as a surfactant and a stabilizer. In some embodiments, the compositions of the present technology comprise CBD and EHG.

In some such embodiments, the compositions of the present technology comprise isopropyl myristate, which is composed of isopropyl alcohol and myristic acid, a common, naturally occurring fatty acid. It is used primarily as an emollient or thickening agent. In some embodiments, the compositions of the present technology comprise CBD and isopropyl myristate.

In some such embodiments, the compositions of the present technology comprise Coco Caprylate (also referred to as Coco Silicone), which is the mixture of esters that occur when the fatty alcohols derived from coconut alcohol react with the fatty acid caprylic acid. It is used primarily as an emollient. In some embodiments, the compositions of the present technology comprise CBD and Coco Caprylate.

In some such embodiments, the compositions of the present technology comprise vitamin E (dl-α-tocopheryl acetate), which is a fat-soluble nutrient often used for its antioxidant and immune system-boosting properties. In some embodiments, the compositions of the present technology comprise CBD and vitamin E.

In some such embodiments, the compositions of the present technology comprise Xanthan gum, which is a soluble fiber primarily used as a thickening or stabilizing agent. In some embodiments, the compositions of the present technology comprise CBD and Xanthan gum.

The compositions of the present technology may comprise an emollient. For example, the emollient may be selected from one or more of the following classes: Triglycerides (e.g. Medium-chain triglycerides (MCT)), Triglyceride esters which include, but are not limited to, vegetable and animal fats and oils such as castor oil, cocoa butter, safflower oil, cottonseed oil, corn oil, olive oil, cod liver oil, almond oil, avocado oil, palm oil, sesame oil, squalene, kikui oil and soybean oil; Acetoglyceride esters, such as acetylated monoglycerides; Ethoxylated glycerides, such as ethoxylated glyceryl monostearate; Alkyl esters of fatty acids having 10 to 20 carbon atoms which include, but are not limited to, methyl, isopropyl, and butyl esters of fatty acids such as hexyl laurate, isohexyl laurate, isohexyl palmitate, isopropyl palmitate, methyl palmitate, decyloleate, isodecyl oleate, hexadecyl stearate decyl stearate, isopropyl isostearate, methyl isostearate, diisopropyl adipate, diisohexyl adipate, dihexyldecyl adipate, diisopropyl sebacate, lauryl lactate, myristyl lactate, and cetyl lactate; Alkenyl esters of fatty acids having 10 to 20 carbon atoms such as oleyl myristate, oleyl stearate, and oleyl oleate; Fatty acids having 10 to 20 carbon atoms such as pelargonic, lauric, myristic, palmitic, stearic, isostearic, hydroxystearic, oleic, linoleic, ricinoleic, arachidic, behenic, and erucic acids; Fatty alcohols having 10 to 20 carbon atoms such as lauryl, myristyl, cetyl, hexadecyl, stearyl, isostearyl, hydroxystearyl, oleyl, ricinoleyl, behenyl, erucyl, and 2-octyl dodecanyl alcohols; Lanolin and lanolin derivatives such as lanolin, lanolin oil, lanolin wax, lanolin alcohols, lanolin fatty acids, isopropyl lanolate, ethoxylated cholesterol, propoxylated lanolin alcohols, acetylated lanolin alcohols, lanolin alcohols linoleate, lanolin alcohols ricinoleate, acetate of lanolin alcohols ricinoleate, acetate of ethoxylated alcohols-esters, hydrogenolysis of lanolin, ethoxylated hydrogenated lanolin, and liquid and semisolid lanolin absorption bases; Polyhydric alcohol esters such as ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol polyfatty esters, ethoxylated glyceryl monostearate, 1,2-butylene glycol monostearate, 1,2-butylene glycol distearate, sorbitan fatty acid esters, and polyoxyethylene sorbitan fatty acid esters; Wax esters such as beeswax, spermaceti, myristyl myristate, stearyl stearate; Beeswax derivatives such as polyoxyethylene sorbitol beeswax which are reaction products of beeswax with ethoxylated sorbitol of varying ethylene oxide content, forming a mixture of ether esters; Vegetable waxes including, but not limited to, carnauba and candelilla waxes; Phospholipids such as lecithin and derivatives; Sterols including, but not limited to, cholesterol and cholesterol fatty acid esters; and Amides such as fatty acid amides, ethoxylated fatty acid amides, and solid fatty acid alkanolamides.

In some such embodiments, the compositions of the present technology comprise sorbitol. In some embodiments therefore, the compositions of the present technology comprise CBD and sorbitol.

In some such embodiments, the compositions of the present technology comprise MCT oil. In some embodiments therefore, the compositions of the present technology comprise CBD and MCT oil.

In some embodiments, the non-medical ingredients to enter into the compositions of the present technology are naturally occurring or are naturally derived or are a combination thereof. As used herein, the expression “naturally occurring” refers to a natural product that is delivered in a natural form. The expression “naturally derived”, as used herein, refers to some ingredients derived from nature that have been used to artificially create a product that is delivered in an unnatural form.

v) Formulation and Method of Use

Cannabinoid receptor agonists (e.g., cannabinoids) have antinociceptive and anti-hyperalgesic effects at the peripheral and central (spinal and supraspinal) levels, as has been demonstrated in acute and chronic pain models. Cannabinoid receptors and endocannabinoids are present in pain circuits from the peripheral sensory nerve endings up to the brain. Cannabinoid receptor agonists modulate nociceptive thresholds by regulating neuronal activity, but they also relieve pain by acting on non-nervous tissues. CB₁ receptor is involved in the attenuation of synaptic transmission, and a proportion of the peripheral analgesic effect of endocannabinoids can be attributed to a neuronal mechanism acting through CB₁ receptors expressed by primary afferent neurons. However, recent findings suggest that CB₁ receptors are also present in mast cells and may participate in some anti-inflammatory effects. Thus, activated CB₁ receptors present in mast cells induce sustained cAMP elevation, which, in turn, suppresses degranulation. On the other hand, although CB₂ receptors have been related traditionally to the peripheral effects of cannabinoids (mainly modulation of the immunologic responses), they also contribute to antinociception by inhibiting the release of proinflammatory factors by non-neuronal cells located near nociceptive neuron terminals. CB₂ receptors are expressed in several types of inflammatory cells and immunocompetent cells. For that reason, activation of peripheral CB₂ receptors generates an antinociceptive response in situations of inflammatory hyperalgesia and neuropathic pain, while selective CB₂ receptor agonists are not antihyperalgesic against chronic inflammatory pain in CB₂ knockout mice. Possible mechanisms of this CB₂-mediated effect include the attenuation of NGF-induced mast cell degranulation and of neutrophil accumulation, both of which are processes known to contribute to the generation of inflammatory hyperalgesia. Therefore, since activation of CB₁ receptors is associated with central side effects, including ataxia and catalepsy, selective CB₂ receptor agonists have the potential to treat pain without eliciting the centrally mediated side effects. Furthermore, CB₂ receptors have novel pain control actions. A CB₂-mediated effect exists, consisting in the indirect stimulation of opioid receptors located in primary afferent pathways. Thus, cannabinoid compounds can modulate hyperalgesia of various origins and they are effective even in inflammatory and neuropathic pain, which are conditions often refractory to treatment.

As such, in some embodiments, the compositions of the present technology are used for managing pain in a subject. In some implementations of these embodiments, the management of pain includes alleviating the symptoms of pain. In some other implementations, the management of pain includes the treatment of pain.

As used herein, the term “pain” includes acute pain, inflammatory pain, visceral pain, breakthrough pain, nociceptive pain, neuropathic pain, chronic pain, and cancer-related pain. Neuropathic pain is initiated or caused by a primary lesion or dysfunction of the nervous system. Neuropathic pain may be classified as peripheral neuropathic pain and/or central neuropathic pain (central pain). Non-neuropathic chronic pain (or chronic non-neuropathic pain, commonly referred to as “chronic pain” in comparison with chronic neuropathic pain, commonly referred to as “neuropathic pain”) is an even greater health problem that afflicts a significant number of patients, resulting in personal suffering, reduced productivity and substantial health care costs. Chronic pain includes back pain, rheumatoid arthritis, osteoarthritis, inflammatory pain, non-inflammatory pain, myofascial pain, fibromyalgia, cancer pain, visceral pain, somatic pain, pelvic pain, musculoskeletal pain and idiopathic pain.

In some embodiments, the compositions of the present technology are used in methods for the management of neuropathic pain. In some embodiments, the compositions of the present technology are used in methods for the management of joint pain. In some embodiments, the compositions of the present technology are used in methods for the management of muscular pain.

In some implementations of these embodiments, the methods of the present technology comprise administering the composition of the present disclosure to a subject experiencing pain. In some instances, the administration comprises applying the composition to the area (or areas) on the subject where pain is experienced.

A “subject” may be a human or an animal, such as without limitation a cat, a dog, a monkey, a mouse, a rat, or a rodent. In some embodiments, a subject is a patient experiencing pain or otherwise in need of the management, alleviation or treatment of pain.

In some embodiments, the compositions of the present disclosure are formulated to a form suitable for administration to a subject (e.g., human, animal). In some implementations of these embodiments, the compositions of the present disclosure are formulated in a form suitable for topical administration to a subject.

As used herein, the expression “topical administration” means application to body surfaces such as the skin or mucous membranes and includes without limitation administration to skin and/or mucosal surfaces, including airway surfaces, transdermal administration, vaginal administration, and other topical routes of administration as are known in the art. The most suitable route in any given case will depend on the nature and severity of the condition being treated. Compositions suitable for topical application to the skin preferably take the form of an ointment, cream, foam, lotion, paste, gel, spray, aerosol, or oil. Additional carriers which may be used to administer the compositions defined herein include petroleum jellies, lanolines, polyethylene glycols, alcohols, transdermal enhancers, and combinations of two or more thereof. In some instances, the compositions of the present technology may be formulated into a patch for application to, for example, the skin of a subject.

In some embodiments, the compositions defined herein, upon delivery to the skin of a subject will be distributed to one or more of the following: epidermis, dermis, hypodermis, hair follicle, sweat gland, fat and connective tissue of the skin. In some instances, the compounds of the compositions will be partly or entirely distributed to the systemic circulation of the body. In other instances, the compounds of the compositions will be distributed in the layers of the skin with moderate to no distribution in the systemic circulation. In yet other instances, one or more compounds of the compositions will be partly or entirely distributed to the lymphatic circulation of the body.

In other embodiments, the present technology relates to kits for use in the management of pain involving the administration of the compositions as defined herein to a subject in need of such pain management. In some implementations, the kit comprises: (i) a dosage form of the composition of the present technology; (ii) a container for the dosage form; and one or more of the following optional components (iii) to (ix): (iii) a container for individual units of the dosage form (e.g., individual patches); (iv) educational instructions in any media about various medical conditions, their etiology, pathophysiology, consequences and treatment, including information on the proper use and disposal of the medication (i.e., the composition/dosage form); (v) containers or bags for the safe disposal of any used or remaining unused dosage form, preferably child proof and/or flushable; (vi) tamper evident and child proof packaging for the kit and its contents; (vii) gloves; (viii) adhesive or non-adhesive dressing or fasteners to cover the site of application; and (ix) a cleansing swab, solution or gel for use before or after the application of the composition/dosage form.

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1—Topical Composition for Muscle Join Pain

The following composition for use in alleviating muscle joint pain was prepared. The following components were combined:
50 mg CBD isolate powder (99% pure);
60 mg purified terpenes (mixture of bisabolol and copaene);
30 mg anti-inflammatory botanical extract; and
790 mg non-medicinal ingredients.

The composition was formulated into a cream for topical application with a viscosity of between about 4500 and 5500 cPs.

### Example 2—Topical Composition for Neuropathic Pain

The following composition for use in alleviating neuropathic pain was prepared. The following components were combined:
50 mg purified CBD (99% pure);
50 mg purified terpenes (mixture of copaene and capsaicin);
40 mg anti-inflammatory botanical extract; and
790 mg non-medicinal ingredients.

The composition was formulated into a cream for topical application with a viscosity of between about 4500 and 5500 cPs.

### Example 3—Topical Cream Composition for Exercise Induced Muscle Soreness and Markers of Inflammation

A double-blind, randomized, placebo-controlled Phase I study will be performed to evaluate the safety, tolerability, and absorbance of topical Cannabis-containing compositions and the relative effects of these compositions on exercise induced muscle soreness and on expression of markers of inflammation in healthy adult.

This study will attempt to: (1) assess safety and tolerability of the active test products compared to placebo (2) if CBD and THC are absorbed systemically over the treatment period, and 3) evaluate the potential efficacy of three Cannabis based (CBD and/or THC) topical analgesics in reducing pain associated with delayed onset muscle soreness (DOMS).

Pain will be assessed during the first 24 hours post-exercise at: 0 (immediately after test product application, T0), 3, 9, 15, 21 and 24 hours, then again at 48 and 72 hours. The following compositions will be tested: Composition #1: High-dose Active Product 1: CBD cream (5% w/w CBD), 3.0 g/application; Composition #2: High-dose Active Product 2: CBD and THC cream (5% CBD, 2% THC w/w or 1.2% THC w/w) 3.0 g/application; and Composition #3: Placebo (same consistency as active product creams): 3.0 g/application.

### Example 4—Topical Nerve Cream Composition

The following topical nerve cream composition was prepared.
5% w/w CBD Isolate (99%);
2% w/w purified terpenes (mixture of copaene and capsaicin);
7% w/w anti-inflammatory botanical extract; and
86% w/w non-medicinal ingredients.

A. Preparation of the aqueous phase: the water soluble ingredients were weighed into a suitable vessel and heated in a water bath while mixing with a propeller stirrer. The mixture was brought to a temperature of 75° C.±2° C.

B. Preparation of the Oil phase: the water insoluble ingredients were weighed into a suitable vessel and mixed well. The mixture was heating on a hot plate using a propeller stirrer at slow speed, to mix/melt the contents and bring the temperature of the contents to 75° C.±2° C.

C. Preparation of Coarse Emulsion: The speed of mixing of the aqueous phase (A) was increased and then maintained at 700 to 900 rpm and the heated oil phase (B) was gradually added into the aqueous phase and mixed at 900 rpm at 75° C.±2° C. to form a coarse emulsion.

D. Homogenization: The propeller mixer was stopped, the hot plate turned off, and homogenization of the coarse emulsion was started at 6000 rpm until the temperature of the emulsion gradually dropped to 50° C.±2° C. Then the homogenization speed was increased to 10,000 rpm and gradually the temperature of the emulsion was dropped to 39° C.±2° C. (cold tap water was added in the water bath to drop temperature gradually). Homogenization speed of the emulsion was reduced to 6000 rpm and the weighed Tazman Pepper™ was added into the mixture and continued homogenizing until the temperature dropped to 33° C.±2° C.

E. Stopped homogenizing and started mixing the product with the propeller mixer at 300 to 400 rpm and used a scraper to scrape product from the sides of the vessel, allowing it to congeal and form a homogeneous product. Product was allowed to cool down to 25° C.±2° C. Product was then transferred into a suitable container.

### Example 5—Topical Muscle Cream Composition

The following muscle cream composition was prepared:
5% w/w CBD Isolate (99%);
6% w/w purified terpenes (mixture of bisabolol and copaene);
8% w/w anti-inflammatory botanical extract; and
81% w/w non-medicinal ingredients.

A. Preparation of the aqueous phase: the water soluble ingredients were weighed into a suitable vessel and heated in a water bath while mixing with a propeller stirrer. The mixture was brought to a temperature of 75° C.±2° C.

B. Preparation of the Oil phase: the water insoluble ingredients were weighed into a suitable vessel and mixed well. The mixture was heating on a hot plate using a propeller stirrer at slow speed, to mix/melt the contents and bring the temperature of the contents to 75° C.±2° C.

C. Preparation of Coarse Emulsion: The speed of mixing of the aqueous phase (A) was increased to 550-600 rpm, and gradually the heated oil phase (B) was added into the aqueous phase and mixed at 75° C.±2° C. to form a coarse emulsion.

D. Homogenization: The propeller mixer was stopped, the hot plate turned off, and homogenization of the coarse emulsion started at 6000 rpm until the temperature of the emulsion gradually dropped to 50° C.±2° C. Then the homogenization speed was increased to 10,000 rpm and the temperature of the emulsion gradually dropped to 39° C.±2° C. (with cold tap water added in the water bath to drop the temperature gradually). Homogenization speed of the emulsion was reduced to 6000 rpm until the temperature dropped to 33° C.±2° C.

E. Stopped homogenizing and started mixing the product with the propeller mixer at 300 to 400 rpm and used a scraper to scrape product from the sides of the vessel allowing it to form a homogeneous product. This was cooled down to 25° C.±2° C. Product was then transferred into a suitable container.

### Example 6—Topical CBD Cream Composition

The following CBD cream composition was prepared:
5% w/w CBD Isolate (99%);
2% w/w purified terpenes (copaene);
5% w/w anti-inflammatory botanical extract; and
88% w/w non-medicinal ingredients.

A. Preparation of the aqueous phase: the water soluble ingredients were weighed into a suitable vessel and heated in a water bath while mixing with a propeller stirrer. The mixture was brought to a temperature of 75° C.±2° C.

B. Preparation of the Oil phase: the water insoluble ingredients were weighed into a suitable vessel and mixed well. The mixture was heating on a hot plate using a propeller stirrer at slow speed, to mix/melt the contents and bring the temperature of the contents to 75° C.±2° C.

C. Preparation of Coarse Emulsion: The speed of mixing of the aqueous phase (A) was increased to 700-900 rpm and gradually the heated oil phase (B) was added into the aqueous phase and mixed at 900 rpm, keeping the temperature at 75° C.±2° C., to form a coarse emulsion.

D. Homogenization: The propeller mixer was stopped, the hot plate turned off, and homogenization of the coarse emulsion started at 6000 rpm until the temperature of the emulsion gradually dropped to 50° C.±2° C. Then the homogenization speed was increased to 10,000 rpm and gradually the temperature of the emulsion was dropped to 39° C.±2° C. (with cold tap water added in the water bath to drop the temperature gradually). Homogenization speed of the emulsion was dropped to 6000 rpm until the temperature dropped to 33° C.±2° C.

E. Stopped homogenizing and started mixing the product with the propeller mixer at 300 to 400 rpm and used a scraper to scrape product from the sides of the vessel allowing it to congeal and form a homogeneous product. This was allowed to cool down to 25° C.±2° C. Product was then transferred into a suitable container.

### Example 7—Topical CBD/THC Cream Compositions

The following CBD/THC cream compositions were prepared:

Composition A:
2.5% w/w CBD Isolate (99%);
1.2% w/w THC Distillate (85%);
2% w/w purified terpenes (copaene);
5% w/w anti-inflammatory botanical extract; and
89.3% w/w non-medicinal ingredients.

Composition B:
5% w/w CBD Isolate (99%);
1.2% w/w THC Distillate (85%);
2% w/w purified terpenes (copaene);
5.5% w/w anti-inflammatory botanical extract; and
86.3% w/w non-medicinal ingredients.

Composition C:
2.5% w/w CBD Isolate (99%);
0.125% w/w THC Distillate (85%);
2% w/w purified terpenes (copaene);
5% w/w anti-inflammatory botanical extract; and
90.375% w/w non-medicinal ingredients.

Composition D:
2.5% w/w CBD Isolate (99%);
0.5% w/w THC Distillate (85%);
2% w/w purified terpenes (copaene);
5% w/w anti-inflammatory botanical extract; and
90% w/w non-medicinal ingredients.

A. Preparation of the aqueous phase: the water soluble ingredients were weighed into a suitable vessel and heated in a water bath while mixing with a propeller stirrer. The mixture was brought to a temperature of 75° C.±2° C.

B. Preparation of the Oil phase: the water insoluble ingredients were weighed into a suitable vessel and mixed well. The mixture was heating on a hot plate using a propeller stirrer at slow speed, to mix/melt the contents and bring the temperature of the contents to 75° C.±2° C.

C. Preparation of Coarse Emulsion: The speed of mixing of the aqueous phase (A) was increased to 700-900 rpm. The heated oil phase (B) was gradually added into the aqueous phase and mixed at 900 rpm keeping the temperature at 75° C.±2° C., to form a coarse emulsion.

D. Homogenization: The propeller mixer was stopped, the hot plate turned off, homogenization of the coarse emulsion started at 6000 rpm until the temperature of the emulsion gradually dropped to 50° C.±2° C. Then the homogenization speed was increased to 10,000 rpm and gradually the temperature of the emulsion was dropped to 39° C.±2° C. (cold tap water was added in the water bath to drop the temperature gradually). Homogenization speed of the emulsion was reduced to 6000 rpm until the temperature dropped to 33° C.±2° C.

E. Stopped homogenizing and started mixing the product with the propeller mixer at 300 to 400 rpm and used a scraper to scrape product from the sides of the vessel allowing it to congeal and form a homogeneous product. This was allowed to cool down to 25° C.±2° C. Product was then transferred into a suitable container.

### Example 8—Permeation of CBD, CBD/THC Compositions

A study was carried out to assess permeation of compositions according to some embodiments of the present technology. In vitro permeation tests (IVPT) of the topical compositions identified in Table 1 were carried out through ex vivo human skin using Franz Diffusion Cell Finite Dosage Model with sample analysis by UPLC-MS/MS according to standard protocols.

TABLE 1
IVPT tested Compositions

| Test | | CBD | THC |
|---|---|---|---|
| Com- | | content | content |
| positions | | | |
| C1 | Composition of Example 4 | 5% w/w | 0% w/w |
| C2 | Composition of Example 5 | 5% w/w | 0% w/w |
| C3 | Composition C of Example 7 | 5% w/w | 0% w/w |
| C4 | Composition D of Example 7 | 2.5% w/w | 0.5% w/w |

The IVPT test system measured the accumulation of the CBD in the receptor solution (systemic circulation mimic) from t=0 hour through t=48 hours after application of the test compositions to the top of the human skin mounted in the chamber. The distribution of the CBD throughout the skin was determined by separation of the skin layers and extracting them after 48 hours of application of the test compositions. The system measured passive permeation as there was no mechanical rubbing of the test compositions into the skin sample and there was no active blood circulation in the skin sample. Results of the IVPT test are presented in Tables 2 and 3.

TABLE 2
IVPT Result Summary

| | Skin Permeation (% Applied Dose) | | | | |
|---|---|---|---|---|---|
| | C1 | C2 | C3 | C4 | C4 |
| | (% CBD) | (% CBD) | (% CBD) | (% CBD) | (% THC) |
| IVPT | 5.7 | 4.3 | 5.3 | 3.7 | 3.4 |
| Skin | 29.15 | 37.05 | 28.2 | 39.9 | 38.8 |
| Skin Strip | 32.2 | 36.1 | 37.9 | 29.4 | 26.7 |
| Skin Wash | 33.05 | 22.6 | 28.8 | 26.9 | 31.2 |
| Total Absorbed | 67.0 | 77.4 | 71.3 | 73.1 | 68.9 |

TABLE 3
IVPT Result Summary

| | Skin Permeation (% Absorbed Dose) | | | | |
|---|---|---|---|---|---|
| | C1 | C2 | C3 | C4 | C4 |
| | (% CBD) | (% CBD) | (% CBD) | (% CBD) | (% THC) |
| IVPT | 8.8 | 5.9 | 8.2 | 7.4 | 7.7 |
| Skin | 42.8 | 45.9 | 37.2 | 51.9 | 54.2 |
| Skin Strip | 48.4 | 48.2 | 54.7 | 40.7 | 39.9 |

All tested compositions provided ˜70% skin absorption without physical assistance. The tested compositions delivered ˜35% of the applied amount of CBD to the dermis and ˜4.5% through the dermis to underlying tissues for microcirculation. CBD was delivered continually over the test period.

### INCORPORATION BY REFERENCE

All references cited in this specification, and their references, are incorporated by reference herein in their entirety where appropriate for teachings of additional or alternative details, features, and/or technical background.

### EQUIVALENTS

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following embodiments.

1. A composition comprising:
a purified cannabidiol (CBD) or a derivative thereof; and
an anti-inflammatory botanical extract comprising Arnica extract and Boswellia extract.

2. The composition according to claim 1, further comprising
at least one purified terpene or a derivative thereof.

3. The composition according to claim 1, wherein the composition is in a form suitable for topical administration to a subject.

4.-9. (canceled)

10. The composition according to claim 2, wherein the at least one purified terpene or the derivative thereof is one or more of: bisabolol, cadinene, caryophyllene or beta-caryophyllene, camphor, capsaicin, carvacrol, copaene, farnesol, guaia-1 (10),11-diene, humulene, isoborneol, γ-linolenic acid, methyl salicylate, β-sitosterol, brassicasterol, stigmasterol, campesterol and γ-terpinene.

11. (canceled)

12. The composition according to claim 2, wherein the at least one purified terpene or the derivative thereof comprises bisabolol.

13.-14. (canceled)

15. The composition according to claim 2, wherein the at least one purified terpene or the derivative thereof comprises capsaicin.

16. The composition according to claim 2, wherein the at least one purified terpene or the derivative thereof comprises beta-caryophyllene.

17. The composition according to claim 1, wherein the purified cannabidiol or the derivative thereof is present in the composition in an amount between about 2 wt % and about 6 wt % of the total weight of the composition.

18. (canceled)

19. The composition according to claim 2, wherein the at least one purified terpene or the derivative thereof is present in the composition in an amount between about 0.25 wt % and about 20 wt % of the total weight of the composition.

20. The composition according to claim 2, wherein the at least one purified terpene or the derivative thereof is present in the composition in an amount between about 0.25 wt % and about 8 wt % of the total weight of the composition.

21.-26. (canceled)

27. The composition according to claim 1, wherein the composition comprises one or more of: Coco Caprylate: Polawax™ NF; Isopropyl myristate; MCT Oil; Ethylhexyl glycerin; Phenoxyethanol; Vitamin E; Polysorbate 60; Copaiba Balsam Essential Oil; Angelica Root Extract; Wild Yam Extract; Polysorbate 80; Span™ 60; Aloe Vera juice; EDTA Disodium Dihydrate; Sorbitol; Xanthan Gum; and Tazman Pepper™.

28. The composition according to claim 1, wherein the anti-inflammatory botanical extract is present in the composition in an amount of between about 0.5 wt % and about 90 wt % of the total weight of the composition.

29.-31. (canceled)

32. A method for management of pain in a subject, the method comprising administering the composition as defined in claim 1 to the subject.

33. A method for alleviating pain in a subject, the method comprising administering the composition as defined in claim 1 to the subject.

34. A method for treating pain in a subject, the method comprising administering the composition as defined in claim 1 to the subject.

35.-38. (canceled)

39. The composition according to claim 2, wherein the at least one purified terpene or the derivative thereof comprises menthol, camphor, or a combination thereof.

40. The composition according to claim 1, wherein the purified cannabidiol or the derivative thereof is present in the composition in an amount between about 1 wt % and about 10 wt % of the total weight of the composition.

41. The composition according to claim 1, wherein the anti-inflammatory botanical extract is present in the composition in an amount of between about 5 wt % and about 10 wt % of the total weight of the composition.

42. A composition for topical application comprising:
a purified cannabidiol (CBD) or a derivative thereof in an amount between about 1 wt % and about 10 wt % of the total weight of the composition; and
an anti-inflammatory botanical extract in an amount of between about 0.5 wt % and about 90 wt % of the total weight of the composition, wherein the anti-inflammatory botanical extract comprises Arnica extract and Boswellia extract.

43. A composition for topical application comprising:
a purified cannabidiol (CBD) or a derivative thereof;
an anti-inflammatory botanical extract in an amount of between about 0.5 wt % and about 90 wt % of the total weight of the composition, wherein the anti-inflammatory botanical extract comprises Arnica extract and Boswellia extract; and
at least one purified terpene or a derivative thereof.
